(19) United States
(12) Patent Application Publication
(10) Pub. No.: US 2009/0221999 A1
(43) Pub. Date: Sep. 3, 2009

(54) Thermal Ablation Design and Planning Methods

(76) Inventor: Ramin Shahidi, Stanford, CA (US)

Correspondence Address: SQUIRE, SANDERS & DEMPSEY L.L.P., 1 MARITIME PLAZA, SUITE 300, SAN FRANCISCO, CA, 94111, US

(21) Appl. No.: 12/040,129

(22) Filed: Feb. 29, 2008

(51) Int. Cl. A61B 18/18

(52) U.S. Cl. 606/33; 128/898

(57) ABSTRACT

Methods for simulation of heat transport phenomena applicable to the design of a near-field microwave ablation device, the design of such a device based on simulation and a patient planning and monitoring station using simulated thermal ablation of tissue are provided.

### RELATED APPLICATION

This application claims the benefit under 25 U.S.C. § 120 of U.S. Provisional Application No. 60/892,124 filed Feb. 28, 2007.

### BACKGROUND OF THE INVENTION

1. Field of the Invention

The invention relates to the field of thermal ablation devices, methods for designing thermal ablation devices and systems for using thermal ablation devices.

2. Background

In 2006, an estimated 1.6 million Americans will be diagnosed with cancer and 1 in 4 deaths will be due to the disease. In addition to the tragic human toll, the cost of fighting cancer exceeds $231 billion annually in the U.S.; of this, over $15 billion is spent on sophisticated products to treat and support cancer patients. The segment forecasted to experience the greatest relative gains over the 2005-2010 forecast period is minimally invasive tumor ablation, specifically cryoablation, radiofrequency and microwave-based techniques in treating certain patient subsets with liver, prostate and breast cancers. Despite their great promise, these techniques are at their infancy. Insufficiently accurate tumor targeting remains one of the major limitations in using any tumor ablation therapy. Current clinical ablation techniques have only rudimentary preoperative planning or intraoperative targeting. The therapeutic area is a fixed spherical (or semi-spherical) thermal shape, with little control over the heat propagation in the tissue. These factors have increased the chances of reoccurrence of cancer, due to the partial treatment of the pathology using such techniques. Some studies have reported up to 40% reoccurrences in liver treatments. The research and development of technologies and dissemination of freely available treatment planning software (discussed in the Data Sharing Plan) involving the controlled ablation of arbitrarily shaped tumors would assist in the future design of more precise and effective treatments.

### RELATED ART

Surgical resection is currently considered the best treatment for hepatic malignancies, but is not an option for the vast majority ˜90% of patients due to factors such as tumor location, operational risk, function organ reserve or coagulopathy. Traditionally, nonsurgical treatment options consisted of chemo- or radiotherapy, which are largely considered palliative rather than curative and produce undesirable systemic effects. Over the past two decades, there has been a great deal of interest in achieving a curative outcome in unresectable cases by use of minimally invasive in situ ablation techniques including percutaneous ethanol injection, high-frequency focused ultrasound, cryoablation, radiofrequency ablation, percutaneous laser therapy and microwave ablation. The goal of these technologies is to completely destroy a tumor via rapid, lethal local infusion (or extraction) of energies or chemicals. Even in cases that maybe candidates for resection, there are many potential advantages that a minimally invasive treatment could offer over a surgical one such as reduced morbidity and mortality, lower cost and quicker recovery times. However, the reliability and efficacy of minimally invasive ablation therapies need to be improved to increase applicability and fully realize these benefits.

In the United States, radiofrequency ablation (RFA) is by far the most widely accepted and developed of these technologies. A probe (or an array of probes) is inserted into the tumor and emits EM waves in the radiofrequency range (around 500 kHz). This produces an alternating current in the tissue which causes Ohmic heating. Typically, the entire volume plus a 1 cm margin is ablated. RF ablation has also been used for non-tumor ablations in the endocardium and cornea.

RFA probes were first described by electrocautery knives modified to pass RF energy into the body instead of simply using it to heat the surgical tip. The region ablated was small, (2 cm) and most efforts since have centered on increasing necrosis size by techniques such as bipolar probes, expandable arrays, saline infusion probes, cooled-tip probes, and pulsed waveforms. Currently, there are three companies that commercially manufacture RFA probes in the United States: Rita Medical and Radiotherapeutics (Boston Scientific) produce expandable multi-probe arrays, while Radionics (Tyco) specializes in pulsed and cooled-tip probes. In perfused porcine liver, current commercial probes can produce thermal necroses of diameter up to 4.5 cm.

There are several key short comings of current RFA systems which become evident upon examination of clinical data. When reported, between 67 and 84% of treatments achieved complete tumor ablation, with tumor size being the most important determining factor. Local reoccurrence rates ranged from 4 to 55%, and complication rate over 82 studies surveyed was 8.9%. There are potential problems with the RFA heating mechanism itself which limits efficacy and can lead to tumor reoccurrence. Exposure to temperatures of 46° C. for one hour is lethal to cells, and much shorter times are needed for higher temperatures (several minutes at 60°). To ensure complete destruction of a tumor, it is necessary to expose the entire volume to a combination of adequate temperature and time. Probes operating in the radiofrequency range are limited by a relatively small zone of actual energy deposition. Most of the actual heating occurs by conduction, which is slow and inefficient in tissue, and particularly problematic around heat sinks such as vasculature. Additionally, the exterior margins of the tumor are the most dangerous regions, and because of reliance on heat conduction these regions are the least efficiently heated in the tumor, especially with single-source probes.

Microwave tumor ablation (MTA) devices operate at frequency ranges of 1 GHz and above. At these frequencies, polar molecules such as water attempt to align with the rapidly oscillating field, and heating is produced by frictional opposition. As a consequence, the zone of direct heating (field heating) is much larger than in RFA, less sensitive to vascular heat sinks, and can be produced more predictably and rapidly. However, due to higher tissue conductivities at microwave frequencies, less total energy is delivered at identical source strengths using a microwave device compared to RFA.

The necessity of inserting a probe into a malignant tumor in both RFA and MTA presents several risks. There is the possibility of track seeding, in which cells from the tumor use the probe introduction path as an avenue to disperse to other parts of the body. If the tumor is highly vascularized, there is a risk of excessive bleeding. To avoid these complications, an extra-tumoral heating device which would not need to penetrate the tumor is desirable. Broadly, limitations on the safety and efficacy of current probes can be traced to three areas: ablation size, ablation geometry shaping and targeting. Over 95% of work to date has focused on increasing ablation necrosis size. While this has allowed treatment of larger tumors, the absence of either spatial or temporal control of heating or guidance contributes to incomplete treatments and complications from damage to unintended structures. Better control of ablation shaping and targeting will enable more precise ablations, especially of irregular tumors, as well as avoidance of structures which would be undesirable to heat. The latter ability is especially important for extra-hepatic ablations sites. The development of shaping and targeting abilities will require advances in energy deposition such as inferential techniques, improvements in image-guidance, as well as better understanding and models of how heat transfer occurs in tissue.

RFA is commonly performed under ultrasound (US) guidance with features such as Color Doppler to monitor hypervascularity in a tumor. In order to achieve proper three-dimensional positioning of an RFA device, the US probe often needs to be manually rotated, which can be cumbersome and highly operator-dependent. The advent of small three-dimensional US transducers will greatly simplify placement. Currently, preoperative images must be manually evaluated to qualitatively determine the spatial location of the tumor relative to blood vessels (heat sinks) or anti-target structures, and the treatment adjusted accordingly. Intraoperatively, microbubble formation in heated tissue can be used to roughly track treatment progress. RFA can be performed in an open procedure, laprotomy or percutaneously. The more invasive approaches are associated with better outcomes, indicating inadequate image guidance and treatment planning necessary for the less invasive approaches. Less commonly, RFA can be performed under magnetic resonance imaging (MRI) guidance. Although MRI provides better contrast and spatial data, it is considerably more expensive than ultrasound and cannot provide real-time data. Treatment must be adjusted since the RF signal from the ablation probe interferes with MRI image acquisition.

Numerical models of ablation can address two distinct physical phenomena: EM-tissue interactions and the resultant redistribution of the deposited heat. Groups have predominantly used finite element methods (FEM) to model heat transfer, and either FEM or finite-difference time-domain (FDTD) to model the electromagnetics. RFA models generally simplify the EM portion, since heat conduction is dominant and tissue can be considered purely resistive at RF frequencies. In the limited MTA modeling literature, a full implementation of Maxwell's equations must be used to adequately model the field heating. Most biological EM-thermal computational models have come from the therapeutic hyperthermia literature. Hyperthermia devices use interference patterns produced by phased-array emitters to selectively heat desired regions within the body. They are distinct from ablation devices in that they produce small elevations in temperature that are not necessarily lethal to tumors, but typically used an adjuvant to other therapies. The energy is deposited in the antenna far-field, as opposed to the near-field as in MTA, an important physical distinction. Efforts in EM modeling for hyperthermia have included adaptive feedback phasing algorithms and broadband energy deposition.

The greatest challenge facing physics-based heat transfer modeling has been the effect of blood perfusion. The widely used bioheat equation (BHE) proposed by Pennes in 1948 makes many simplifying assumptions including temperature-independent tissue properties. Tompkins et al in 1994 noted that perfusion in normal tissues increased with temperature, while some tumors showed an opposite response. Perfusion rates in hypo- and hyper-vascularized tumors are vastly different, a phenomena few model have adequately explored. The original BHE formulation also assumes that heat transfer would reach an equilibrium in the microcirculation (allowing perfusion to be lumped into a single term), ignoring convective effects of large vessels. Several RFA and hyperthermia models that have explicitly examined the effects of discrete vessels have clearly demonstrated they have a major impact on heating, and this effect has also been shown experimentally. The local effect of large vessels on microwave ablation has yet to be adequately explored numerically. Accurate time and temperature dependence of electrical and thermal characteristics of biological tissue is also an important research objective, as is simulating an accurate thermal damage function for different tissue types.

Studies have compared their computational results against heated tissue-equivalent phantoms, ex-vivo tissue and in-vivo tissue. Heat transfer can differ markedly between in-vivo and ex-vivo environments since there is generally no perfusion in the latter. One study found that a range of commercial RFA probes produced ablations of volume 55-68 cm³ in ex-vivo liver tissue, but only 29-42 cm³ in in-vivo liver. Attempts have been made to address this problem by constructing phantoms containing a tube through which fluid flows, but this may not capture the effects of microcirculatory perfusion. Assessment of heating effects is typically performed either by temperature probes, which allow measurements over time but only at discrete points, or histologically in tissue, which gives better volumetric information, but only at the end point. A very limited number of groups have explored proton resonance frequency (PRF) MRI thermometry, which can temporally record high-resolution, tomographic, temperature profiles, and would potentially be a powerful tool for model validation.

Most models have quantified the therapeutic affect of heating by reporting either temperature isotherms or thermal dosing as ablation necrosis boundaries. Physiologically however, thermal tissue damage is a function of both time and temperature, and tissue damage affects material properties. Only one group has attempted to examine this thus far, and was able to predict necrosis boundaries with 5% error compared with experiment. Literature on RFA modeling is sparse, and there are even fewer modeling studies that examine ablation using microwave energies. The continued development of robust, validated, field-coupled models is essential to advance treatment planning, precision and better patient outcomes.

### SUMMARY OF THE INVENTION

The invention is directed to processes, methods and systems for simulation and/or prediction of heat transport phenomena within an anatomical landscape. The invention is also directed to processes, methods and systems for increasing the effectiveness of thermally-induced cell necrosis methods through simulation and optimization of probe parameters, thereby increasing the effectiveness of thermal treatment for a target within the body, e.g., a tumor. This can lead to higher success rates, reduce the number of treatment sessions, and reduce side-effects associated with thermal treatment. In view of these teachings, it will become apparent that medical costs associated with thermal ablation treatment can be significantly reduced by the methods, systems and processes of the invention.

According to one embodiment, a method for providing a health professional with a patient-specific thermal ablation tool comprises the steps of constructing mathematical site models simulating heat transport phenomena within the body and then validating each constructed site model, including the step of validating each model against constructed phantoms, and providing the health professional with a machine based routine for predicting the progress of a patient's thermal ablation session using a simulation based on one or more of the validated site models.

According to another embodiment, a thermal therapy tool provided on computer-readable media includes a library of generalized site models, wherein each site model includes a mathematical representation of energy absorption and dissipation characteristics of various inhomogeneous tissue and/or anatomic structure within the body; a first routine constructing a patient-specific model from one or more library models based upon patient-specific input parameters, the patient-specific parameters reflecting the nature of, and relationship among anatomical structures proximal to a site targeted for thermal therapy; a second routine for simulating a thermal response at the targeted site, the routine receiving as input one of a plurality of user-selectable energy sources and the patient-specific model, and a third routine for generating a visual representation of the predicted thermal response within a patient for thermal ablation planning and/or monitoring.

According to another embodiment, a near-field interferential microwave ablation system includes a probe comprising a plurality of antennas for generating an energy pattern based on near-field interferential microwaves, a controller for modifying the phase and frequency of one or more of the antennas, and a planning station for the probe comprising an algorithm for shaping the energy pattern based on a desired thermal treatment profile.

According to another embodiment, a method for monitoring the thermal therapy applied to a diseased site within a patient's body includes the steps of providing a patient-specific heat model for predicting the energy absorption and dissipation properties of inhomogeneous tissue characteristic of the diseased site, providing as input to the patient-specific heat model the treatment parameters including the type of device being used to supply energy to the diseased site, and contemporaneously simulating the thermal response at the diseased using the heat model.

According to another embodiment, a method for monitoring a thermal ablation procedure includes the steps of selecting or defining a set of parameters reflecting at least a thermal sensitivity, heat sink and heat source property of anatomical structure, computing from the parameters a scoring for assessing the progress of the thermal ablation procedure, and displaying a real-time depiction of the degree of tissue necrosis state relative to a device-specific probe.

According to one embodiment, a thermal ablation simulation and patient-specific planning tool is applied to an interferential microwave probe system. For example, the disclosure provides methods for the development and implementation of controlled thermal-ablation of arbitrary shapes within human tissue using interferential microwaves. According to this method, controlled dielectric heating of tissue by microwave energy (due to the saturation of polar molecules within human tissue) is accomplished by focusing near field microwave radiation through interferential techniques.

According to another embodiment, there is a method of correlating patient-specific data to a pre-defined Atlas model of a patient, where the Atlas model is represented by, or derived from a database of models and/or patient data that are averaged to form the Atlas model. The database may include mathematical models, patient-specific data, in vivo data, in vitro data, data based on simulations using structure that mimics anatomy, or a combination of these data types.

It has been recently discovered that near field microwave radiation can be precisely controlled by varying the spatial characteristics of the electromagnetic (“EM”) fields. By controlling the EM, the shape and volume of the area being ablated can be controlled as well. Recent studies have indicated that near-field EM fields can be used to heat selective areas of tissue up to 60° C. (adequate to kill cells in 4-6 minutes) using multiple high-power antennas emitting frequencies between 1-10 GHz. Examples of these types of devices are discussed in greater detail in U.S. Publication Nos. 2005/0209661 and 2005/0205566.

In accordance with one or more of the stated objectives, a simulation technique for ablative planning and probe design for a near-field interferential microwave (“IFM”) probe is validated with phantoms that incorporate the complex nature of, e.g., tumors and surrounding tissue and incorporate the effects of heat propagation as a function of time through a physiological environment. A model validation approach, where increasing complexity is added as less complex simulations are correlated, is adopted to address the complex nature of heat phenomena.

A “phantom” is intended to mean a body of material(s) that replicates the thermal properties of anatomical structure(s), e.g., organ, vascular body, muscle, tendon, nerve, etc. A phantom may refer to a single, essentially homogenous body of tissue, e.g., a fat cell, or a complex body that accounts for blood perfusion effects. i.e., heat transport effects caused by microcirculation. A phantom includes bodies of material whose thermal properties can be highly temperature-dependant. A phantom, like that of tissue found in the body, includes bodies of material whose thermal dissipation or propagation properties change when the temperature increases, such as when a temperature is reached that kills cells. A phantom may include both tissue-simulating material and actual tissue. A “phantom model” is a model intended replicate the area or structure within a body that is also represented by a mathematical model. A phantom model is used to validate a simulation using a math model and may include both artificial and ex vivo anatomical bodies or tissue.

In accordance with one or more of the stated objectives, the disclosure includes an architectural and algorithmic framework enabling the design of a robust image-guided ablation technique that incorporates predictive physiological and anatomical models. For example, in the case of an IFM probe model, not overly simplified and validated electromagnetic (EM)/thermal models according to the disclosure provides key guidance on the electrical and thermal effect on tissue subjected to microwave energy. This will enable new technologies previously thought not achievable due to the complex heat transport processes that occur within the human body. Thus, the disclosure presents a tool that explains, e.g., how multiple antennas emitting above 1 Ghz microwave energy can be phased near-field to yield a desired ablation shape that could subsequently heat an arbitrary volume, e.g., a tumor, localized by an intraoperative image-guidance system, such that a degree of accuracy of 1 centimeter or less is obtained. This particular objective may be realized through the following three-step approach:

1) Development and validation of a heat transport model in biological tissue. A 4D (space & time) numerical model is developed using, e.g., advanced Finite Element Methods (FEM) that quantify the heat propagation from a “thermal energy source” on phantoms of increasing complexity with varying levels of inhomogeneity in tissue characteristics. These models are validated (and calibrated) by running the same experiments under Magnetic Resonance Thermography (MRT) of corresponding phantom models.

2) Advanced electromagnetic simulation development and integration of this model with the thermal model. Using the calibrated math model, investigate the interferential variations of “multiple microwave sources” affecting geometrical shape and degree of heat dissipation in the focal region. With this kind of data available a prototype interferential focused microwave (“IFM”) device can be made.

3) Validation of the integrated electromagnetic—thermal model using a fabricated system Prototype. Using the same experimental protocols and sequencing in phantom development as in Step 1), the IFM simulation tool can be validated by comparing simulation results with those obtained from the prototype system under MRT.

In accordance with one or more of the stated objectives, it is understood that the three-step process outlined above is not limited to IFM probe design, but may easily be applied to the design of other probe types, such as traditional Radio frequency (“RF”) and far-field microwave probes, as well as ultrasound (“US”) probes.

In accordance with one or more of the stated objectives, a method for rapid simulation is provided, which uses a reduced vector space for computing qualitative information about the progress of ablation during, or prior to a patient session. The parameters used to compute this information for a health professional are derived from validated math models, experimental data and/or anonymous patient data sets.

In accordance with one or more of the stated objectives, validated 4D (space & time) numerical models, accounting for heat transport phenomena through a complex anatomical landscape are incorporated into a planning tool for health professionals. In this aspect of the disclosure, validated numerical models of anatomical structures are constructed and validated using a systematic approach, maintained in a library and then accessible to construct patient-specific site models representing the heat dissipation and propagation characteristics of a site within a patient undergoing thermal therapy. According to this aspect, images of the patient are used to construct a mapping of the anatomical structures of the site where the thermal therapy is intended. The mapping is constructed from the validated thermal models. Then, this mapping is morphed to the specific volumetric and spatial relationships among the anatomical structures appearing in the image. From this process a patient-specific site model is produced for near-real-time monitoring of the thermal ablation or ablation planning.

According to another embodiment, probe design and point of care thermal therapy planning is provided through process and method for producing reliable thermal propagation models that form the basis for finding optimal probe parameters and patient-specific heat transport models.

According to another embodiment, an approach for determining antenna configuration and input parameters to produce controllable energy deposition patterns of controllable, therapeutically useful geometries is provided using a validation process and mathematical simulation environment. The therapeutically useful shapes are characterized by the absence of auxiliary hotspots, relatively homogeneity and well-defined borders.

According to another embodiment, a scheme for probe design and thermal ablation planning is provided by the integration of EM and thermal math models.

According to another embodiment, a software program, pre-operative, and/or inter-operative planning system, process and method for health professionals is provided by an ablation prediction algorithm derived from a simulation algorithm derived from and correlated with complex simulation models with a <10% correlation or margin error. According to this embodiment, health professionals are able to run simulations and optimize treatment parameters without requiring numerically-intensive computations.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIGS. 1A, 1B, 1C depict power patterns in a homogeneous media at distances of 0 mm, 5.9 mm and −5.9 mm from a microwave energy source.

FIG. 2 depicts a process of model validation that gradually increases the complexity of the anatomic landscape as less complex models are correlated to phantoms.

FIG. 3 is a perspective view of a phantom used to validate a math model.

FIGS. 4A-4B are renderings of a patient site during treatment of a tumor.

FIG. 5 are channels providing qualitative information about the thermal ablation procedure depicted in FIGS. 4A-4B.

FIG. 6 is a schematic showing one embodiment of a thermal planning station and ablation probe system.

FIG. 7 is an example of a Graphical User Interface (“GUI”) for probe selection and patient-specific model building.

FIG. 8 is a flow diagram depicting various processes discussed in connection with FIGS. 1-7.

FIG. 9 is a depiction of a near-field MW probe for non-invasive liposuction.

FIG. 10 is a schematic of a system associated with the probe depicted in FIG. 9.

### DETAILED DESCRIPTION OF THE INVENTION

According to a first aspect of the disclosure, a model development and validation process for simulation of heat transport phenomena within the body is disclosed. These validated models are then used to develop a near-field microwave (“IFM”) device for thermal ablation of tumors. The validated models are also used to provide a patient planning and monitoring station which can provide near real-time feedback on thermal therapy, and optimization of device-specific probe parameters based on validated simulations and patient-specific anatomy.

### Modulated Near-Field Microwaves Modeling

Since biological tissue may be considered a good conductor at MW frequencies, electromagnetic energy rapidly attenuates due to the small skin depth δ of tissue where δ≈(πfμδ)^{-1/2} (skin depth is the distance in a good conductor at which the fields drop off to ≈37% of their initial value). Since higher frequencies, in general, correspond to higher spatial accuracy, near field energy offers greater accuracy of thermal deposition. For instance the value of skin depth for liver taken from at a frequency of 1 Gz with σ≈1, and ε≈80, is approximately δ≈1.5 cm at body temperature. Pulsed or continuous power heating may be used for cell necrosis. The idea of tumor ablation is to heat the tumor cells and kill them as quickly as possible without damaging the surrounding tissue. However, since much of the heat transfer is through conduction, the tissue response may benefit more from high power pulsed heating as opposed to continuous heating by minimizing collateral damage to surrounding tissue.

Testing of the efficacy of using near-field focused microwaves for a controlled thermal-ablation of biological tissue may begin with the development of an electromagnetic simulation tool based on a Finite-Difference Time Domain (“FDTD”) method. This method may be used to calculate energy deposition in tissue due to phased harmonic microwave sources. The focusing of electromagnetic energy relies on the near-field interference patterns of electric field sources which, in this case, are operated at integer multiples of a chosen fundamental frequency and appropriately phased in order to produce a stationary hot-spot at the desired target. The methodology of this work is based in-part on the work described in U.S. Publication Nos. 2005/0209661 and 2005/0205566.

The FDTD technique may be used to model electromagnetic wave phenomena in complex inhomogeneous media, such as the human body. Each layer of tissue is assumed to be a lossy dielectric material and is completely specified by its corresponding frequency independent electrical properties ε and σ which are the permittivity and conductivity respectively. The electrical properties of the different tissues may be obtained from readily available data sets generated through MRI and other imaging technologies with millimeter resolution.

The heating of the tissue layers is determined by the Specific Absorption Rate (SAR) with SAR=σ, |E|²/2ρ, where σ and ρ are the conductivity and density of the biological tissue respectively and |E| represents the magnitude of the electric field. The SAR represents the amount of EM energy absorbed at a particular region of the body. This quantity can be ascertained from the simulation results allowing for complete control over the selective heating of the tissue being ablated. Through proper adjustment of the amplitude and phasing of each of the sources, near-field energy can be focused into a well characterized shape in the body. The advantage in utilizing harmonic frequencies is that it allows for the formation of a stationary hot spot relative to broadband excitation which produces blurring of the focal region as a consequence of the additional frequency content.

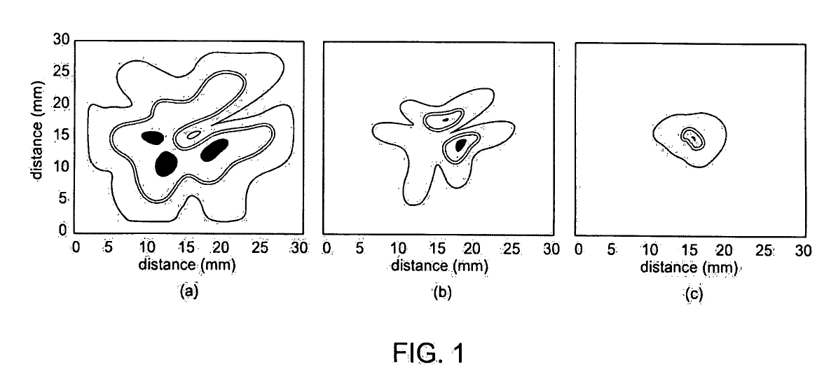

As a point of illustration, the EM interference pattern of a 5 mm diameter probe consisting of four radiating elements using a combination of 5 GHz and 10 GHz frequencies was simulated. FIGS. 1A, 1B and 1C depict the power pattern of three slice planes representing depths below and above the probe. It is seen in FIG. 1A that most of the energy from the probe is focused in the forward direction (z>0) with only minor leakage in the reverse direction. This is due to proper phasing of the antennas.

Designing an ablation tool capable of producing an energy deposition pattern suited for the targeted ablation of an arbitrary-sized tumor, however, first requires a means for understanding the highly complex heat transport and EM energy absorption characteristics of the body necessary for the successful design of an IFM probe. Accordingly, the design of an ablation IFM probe (or more generally an improvement over other probe types) begins with the creation and validation of models that can predict the propagation of heat through the body without over-simplification. For example, it is in general necessary to account for such effects as temperature-dependant heat absorption and/or dissipation characteristics of tissue. Thus, one aspect of the disclosure is the method for creating and then validating a thermal model that incorporates the effects of such non-linearities. Once these validated models are obtained, the spacing and number of antennas, phasing, and frequencies of EM radiation for the ablation tool necessary to produce arbitrary “hot spots” may be found. It will be appreciated that the model creation and validation techniques are applicable to design improvement for other probe types.

### Development and Validation of Thermal Models

A thermal model begins with an assumed thermal deposition pattern. The bio-heat transport equation is given by Eq. (1):

$${C}_{p} ( r ) ρ ( r ) \frac{\partial T ( r )}{\partial t} = ∇ \cdot ( K ( r ) ∇ T ( r ) ) + {A}_{o} ( r ) + Q ( r ) - B ( r ) ( T ( r ) - {T}_{B} ) \qquad ( 1 )$$

where Cp is the specific heat, ρ is the density of the tissue, K is the thermal conductivity, Aₒ is the metabolic heat production, Q is the heating potential related to the SAR, B represents heat exchange with the blood, and T_{B} is the blood temperature. Effects such as blood perfusion are taken into account through B. In the context of EM ablation devices including RFA, MTA and an IFM system, the power delivery mechanism described by Q is included through the addition of Maxwell's equations governing the flow of EM energy.

In order to arrive at a math model consistent with the stated objectives, there is the requirement for modeling small scale and intricate geometries and in homogeneities inherent in body tissue. To this end, a Finite Element Model (“FEM model”) approach may be used to accurately capture heat transport phenomena. A commercial FEM package such as COMSOL may be used. The COMSOL package has Computer Aided Design (CAD) capability, an integrated thermodynamics solver, and the ability to specify arbitrary differential equations, which provides flexibility in the event it is desired to expand a model to account for additional physiological effects on heat transport.

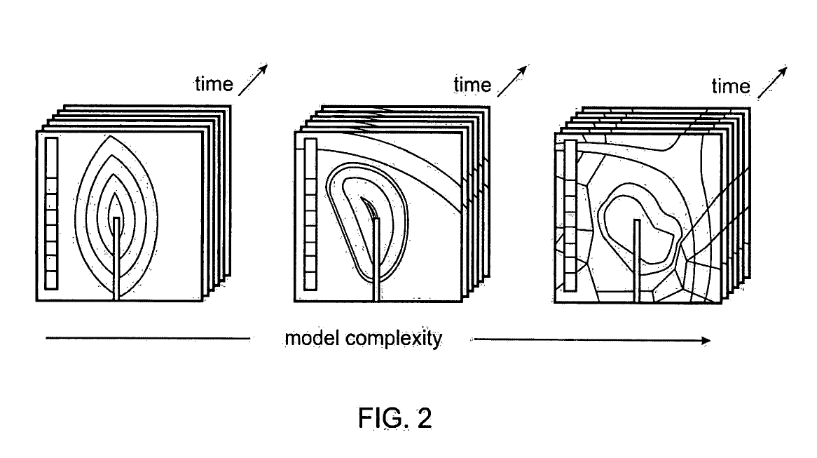

One approach to model validation would be to simulate heat transport from an RFA device. Since RFA probes are ubiquitous and there exists a wealth of data on their performance in clinical application, simulation of RFA devices would be a good approach for model validation. In one embodiment, the simulation environment for a math model of anatomical structure is a phantom of the anatomical structure and MRT. Heat deposition may be initiated using direct application of thermal-coupled RF probes within the phantoms. A design and fabrication of phantoms and MRT protocol for validation purposes is discussed in greater detail, below. The model creation and validation process may proceed by performing repetitive cycles of simulation design, phantom development and MRT studies with the complexity of the simulations, i.e., the math models and associated software routines, and phantoms increasing in complexity as less complex models are successfully correlated. This approach to correlating a 4D model is depicted in FIG. 2. From right to left, the simulation progressively incorporates more in-homogeneity as the less complex simulations are correlated with their corresponding phantoms.

Temperature may greatly effect the thermal properties of tissue thereby augmenting characteristics such as perfusion rates with the inclusion of biological effects such as vasodilatation and cell necrosis. To account for this, there is both an inherent time and temperature dependence assumed for all terms of the bio-heat equation. As such, and consistent with a desire to avoid overly simplifying the model, feedback mechanisms may be included through additional equations that account for a time and temperature dependency affecting the solution of the bio-heat equation. It is necessary, however, to begin the validation scheme with the simplest possible model and then advancing in incremental steps.

There are few data sets in the art for describing the aforementioned tissue dependencies. However, it is understood that these data sets can be assembled without further explanation. Additional systems of equations based on conservation laws (mass, momentum) may be one approach to more accurately model the heat transfer process. See X. Min and R. Mehra. Comparison of methods in approximation of blood flow infinite element models on temperature profile during rf ablation. In Engineering in Medicine and Biology Society, 1998. Proceedings of the 20th Annual International Conference of the IEEE, pages 259-262 vol. 1, 1998; and M. K. Jain and P. D. Wolf. A three-dimensional finite element model of radiofrequency ablation with blood flow and its experimental validation. Annals of Biomedical Engineering; Annals of Biomedical Engineering, 28(9):1075-1084, September 2000. LR: 20041117; PUBM: Print; JID: 0361512.

There are certain thresholds at which tissue damage is irreversible and thus the properties of the tissues will be permanently altered. This nonlinear phenomena is generally more applicable to RFA systems in which temperatures can be in excess of 100° C. However, they may be considered as not a significant contributing factor for a MW ablation devices because they do not require such high temperatures. This is a direct result of the difference in the heat generation mechanism. RF probes rely on direct Ohmic heating and conduction for heat transport, while the higher MW frequencies heat more evenly through capacitive coupling (friction). However, since temperature dependence is generally speaking an important effect to model at all frequencies, it is preferred that the model include non-linear effects of temperature change.

### Electromagnetic Simulation Development and Thermal Model Integration

After validating a model, the next step is to move from an assumed heat deposition pattern to a heat deposition pattern that is a function of the spatial characteristics of EM or other energy source types, e.g., US. For example, in the design of the IFM device, the next step is to integrate an EM model (for the analysis of near-field focusing of IFM devices) into the validated thermal model so that a heat transport process can be predicted based on a specified energy deposition profile.

An understanding of the therapeutic affect of controlled EM energy deposition using an IFM probe requires the successful modeling of both electromagnetic and thermal effects. Upon validation of such a model a design and synthesis of an IFM ablation system prototype becomes possible.

The antenna design is crucial for focusing EM radiation required for controlled tumor ablation. As alluded to earlier, it is possible to focus near-field microwave energy using phased antennas without significantly affecting tissue between the instrument and ablation region. This is illustrated in FIGS. 1A-1C. In order to create an arbitrary shape suited for targeted thermal therapy, the integrated EM/thermal model is needed to determine the exact antenna geometries and excitation characteristics (frequency, amplitude, phasing) that can produce a desired shape for a hot spot.

An integrated EM simulation tool may be based on the FDTD method. However, time domain simulation tools such as FDTD typically require extensive run times making the issue of finding optimal amplitude and phasing parameters for the antenna array within the IFM probe a challenge. According to the disclosure, a more efficient method for determining optimal antenna characteristics uses a frequency domain approach. This technique involves replacing Maxwell's equations with their time-harmonic form. Once a solution is found at a particular frequency for a given antenna, frequency domain linearity and superposition principles are exploited to readily calculate variations of amplitude and phase in a post-processing step on each of the antennas provided by previous simulations without the burden of performing additional time domain runs.

The focusing of microwave energy is obtained through amplitude and phasing adjustments of each antenna element comprising the array. Emphasis is placed on maximizing the energy deposition at the foci and suppression of energy away from target location. Blurring or distortion of the focal point due to the dispersive characteristics of the medium will be treated with the inclusion of additional frequency content and is readily modeled using a frequency domain approach. A Narrowband (NB) source to begin with, followed by a transition to Broadband (BB) may be needed since BB excitation creates added complexity in both equipment and circuit design, but provides more overall flexibility.

In addition to the proper amplitude and phasing of the antennas, the accurate representation of the dielectric properties of the tissue (phantom inclusive) are mandatory for the validation of any simulated results. The electromagnetic properties of biological tissue are in general both frequency and temperature dependent. In order to address the latter, the frequency dependence of the biological tissue may be represented by assuming that the tissue is well represented by a single pole Debye material. With this addition, Maxwell's equations may be solved in conjunction with an auxiliary current equation to account for the frequency dependence. The new system of equations represented in the time-domain are given below:

$$\begin{matrix}∇ x \vec{H} = {ɛ}_{O} {ɛ}_{∞} \frac{d \vec{E}}{d t} + σ \vec{E} + {\vec{J}}_{d} & ( 2 ) \\ ∇ x \vec{E} = - {μ}_{O} \frac{d \vec{H}}{d t} & ( 3 ) \\ {\vec{J}}_{d} + τ \frac{d {\vec{J}}_{d}}{d t} = {ɛ}_{O} ( {ɛ}_{s} - {ɛ}_{∞} ) \frac{d \vec{E}}{d t} & ( 4 )\end{matrix}$$

where \vec{J}_{d} represents the currents in the biological tissue due to the Debye pole, ω is the radian frequency, εₛ is the permittivity of the tissue at zero frequency, ε^{∞} is the permittivity at infinite frequency and τ is the relaxation time constant. The temperature dependence of the material properties can likewise be included with auxiliary equations specific to the material involved.

### Electromagnetic-Thermal Model Integration and Prototype Simulations

As mentioned earlier, integration of both the thermal and electromagnetic models can assist in the design and development of a prototype ablation system. According to one embodiment, a prototype IFM system is developed to validate a IFM simulation model, which then forms the basis for future development of more complex IFM systems.

The computational domain of the integrated EM/thermal model is determined from the smallest scale features present, i.e. vessels, instruments, and then by the characteristic wavelengths in the system that are dominated by the high spatial frequencies of the EM deposition pattern. Thus a grid must capture biological factors, hardware geometries, and correctly sample the smallest EM wave-lengths in the system. Merging two computation grids is a common practice and can be accomplished through known interpolation schemes. A second consideration is the temporal variance in the two simulations. The EM time scales are orders of magnitude smaller than the thermodynamic counterpart. To solve them simultaneously can be computationally intensive and unnecessary. Since the EM power reaches its target instantaneously relative to the time scales over which the heat transfers to the surrounding tissue, the EM portion of the simulation and changes in the EM constitutive parameters can be simplified to a function of some deviation from the baseline without a loss in accuracy.

### Validation of Electromagnetic-Thermal Model and Prototype IFM Probe

The procedure for system validation described earlier assumed a heat deposition pattern. Under an integrated EM/thermal model the heat deposition pattern is no longer assumed. Instead, the simulation and validation requirement now begins with only a pre-defined EM field. As such, the validation process includes the calculation of time and frequency dependent material coefficients appearing in the system of EM equations. Validation, as before, may proceed with a simple homogenous phantom which is gradually increased in complexity. Proton-resonance frequency shift MRT is affected by changes in the electrical conductivity of tissue by as much as 28%. Thus the phantom and simulation should account for the sensitivity with temperature of the electrical properties on any MRT measurements.

At the outset, it is understood that the modeling and validation of physiological processes, and validation of prototype devices in accordance with the stated objectives assumes that there are many unknowns encompassing a wide variety of physics. However, the described incremental development strategy, starting with simplified models and assumed heating patterns, and then increasing in complexity is believed to provide a sound basis for identifying problems in simulation and validation early on, understanding these complexities, and providing a sound basis for making modifications, and without oversimplification of the problem presented. Thus, by utilizing the disclosed methods a more full understanding of complex heat transport processes is gained incrementally. For example, an IFM probe validation is initially based on its operation at low power levels and with homogenous phantoms so that it is safe to assume that the thermal affects on the constitutive properties is negligible. Comparisons should then be relatively straight forward. Likewise, using homogenous phantoms at the outset, validation of the material properties and antenna characteristics will be simplified.

### MRT Protocol and Phantom Development

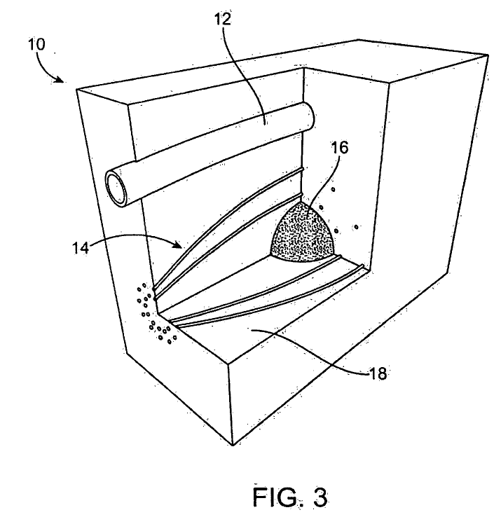

Design and fabrication of phantoms to validate simulation tools may follow that described in K. I Ito, K. Furuya, Y. Okano, and L. Hamada. Development and characteristics of a biological tissue-equivalent phantom for microwaves. Electronics and Communications in Japan (Part I: Communications), 84(4):67-77, 2001. These phantoms, which are electrically equivalent to their biological counterparts within the microwave frequency range consist of deionized water, polyethylene powder, agar, TX-151, sodium chloride (NaCl) and preservatives to maintain their longevity. The electrical characteristics of the phantom are adjustable through variations in the mixing ratios of the ingredients and will maintain consistent electrical properties for more than one month at room temperature. An MRT protocol may involve complex phase-difference mapping based upon the shift in the proton resonance frequency (PRF) in order to monitor temperature propagation through the phantom using MRI. The phantom may be placed in a MRI instrument and subjected to pulsed heating through a direct probe with scans taken in between pulses. This technique is accurate to within 2° C. with spatial resolution of 1 mm providing real-time feedback on the order of 15 s. The imaging parameters that may be used are TE=20-30 ms, TR=150-180 ms, flip angle=60°, FOV=16 cm, 0.75 FOV in the phase encode direction and acquisition matrix of 256×128. MRT may be used for the analysis of the heat deposition characteristics when volumetric temperature data is necessary for development of a planning station and comparison with more advanced phantom design involving heat dissipation mechanisms and inhomogeneous tissue. Cooling effects such as perfusion and the presence of large vessels will be included in the advanced phantom designs by means of a cooling system. On example of a phantom 10 is depicted in FIG. 3.

Phantom 10 includes a cast tube 12 that represents a phantom large vessel, e.g., a primary vein or artery, an array of cast channels 14 representing a phantom microcirculation, a tumor phantom 16, and surrounding tissue phantom 18. The large vessel phantom 12 may be molded from the same material as the surrounding tissue phantom 18. This can prevent the cooling system from acting like an unphysical boundary or scatterer in the thermal and electromagnetic cases respectively. In some cases, the phantom is constructed from artificial material as described above. In more complex phantoms, ex vivo bodies, e.g., Liver tissue, may be included to simulate real tissue.

A validated simulation of anatomical structure, whether as a thermal simulation with assumed heat deposition pattern only, or as an integrated thermal and EM model that takes as input probe parameters, may be integrated into a stand-alone platform for thermal planning and monitoring. According to a second aspect of the disclosure, this platform includes an algorithm, correlated by models, experiments and/or image sets, that enables rapid computer simulation at the point of care.

Geometries of lesions produced by ablation probes can be complex, with parameters such as asymmetrical distortion due to blood vessels and starburst affects in multi-prong probes. Currently, ablation probes are characterized very primitively. Most papers simply report the diameter of lesion produced, with some reporting major and minor axes for oblong lesions. Often, this is the only quantitative data on which treatment planning is based. Blood vessels and inhomogeneous tissue are compensated for qualitatively based largely on operator experience. For large tumors, multiple ablation applications may be necessary to encompass the entire volume, with probe placements for these multiple ablations currently based on the operator's estimates. Clearly, treatment planning tools are needed. Physics-based simulations typically have high computational costs which limits their utility for patient-specific treatment planning, and especially for optimization routines.

### Rapid Simulation of Heat Transport Phenomena

According to a second aspect of the disclosure, there is a method for rapid simulation. Hence, a simulation for use at the point of care is disclosed, which does not rely upon a continuous solution of complex math models. The planning system is based on a library of validated models but need not rely on a computationally-intensive solution when a planned treatment is modified. The planning system may be interactive with image guided selection of anatomical structure, instrument and patient parameters, etc.

One aspect of this planning station is to provide insight to a health professional with how heat will propagate and produce a lesion in a patient-specific anatomical landscape. The approach may be formulated as follows: discretize a treatment space into 3-dimensional Cartesian voxels. Each voxel is decomposed into a vector quantity <ε, K, O>, where ε represents tissue sensitivity, K represents local heat sink affect and O represents energy input from the ablation device. The aggregate over all voxels of each of these components (channels) is referred to as the ε-, K- and O-fields. Tissue sensitivity (ε) quantifies how easily a given voxel is affected by the treatment, and can be determined by a variety of factors. According to this approach, accuracy of the simulation is expected to improve over other approaches because there is no a priori assumption that a probe will always produce a lesion of fixed diameter. Some examples follow:

Different tissue types can be segmented out of an imaging study and assigned different values. For example, adipose tissue is less responsive to heating, while some tumors may be more responsive than the surrounding tissue.

The placement of a grounding pad in an RF ablation can offset the centroid of the thermal lesion. This can be modeled by introducing a gradient in ε.

The ε-field can account for affects at the microcirculation level. Occlusion of the hepatic artery and portal vein results in larger lesions produced, even at sites non-adjacent to those vessels. This can be attributed to a reduction in overall tissue perfusion at the microcirculation level.

Local heat sink affect (κ) accounts for the convective heat loss near large vessels. Each segmented vessel can be modeled to produce a contribution to the κ-field in its vicinity, which would vary as a function of the distance to the center-line of the vessel. The exact relationship may be assigned for each vessel based on simulations, taking into account factors such as occlusion state, probable flow rate, and flow angle and vessel size. Homeostatic response to elevated temperatures via vessel dilation could also be accounted for by changing the κ- and/or ε-field with time. This is an improvement over previous methods because it allows vessels of different characteristics to have a more dynamic range of affects.

Energy Input (O) can be a time-stepped voxel intensity map of the general energy deposition pattern of the probe. It will depend on type, location, orientation and input and driving parameters of the ablation probe. This can be derived from an FEM simulation of the lesion produced by the probe in a homogenous environment. One advantage of this approach is that it is modality-independent, capable of modeling RF, microwave and possibly other thermal ablation techniques such as laser or high intensity focused ultrasound. For example, an RFA probe O-field would begin as high-intensity points at the tip, and expands with time. Regions of highest intensity would still remain concentrated near the tip over time, simulating reliance on conduction (essentially a point source). Multi-prong probes could be modeled using multiple point sources. MTA field heating may behave differently. MTA heating could be expressed as an O-field that resembles a plateau with respect to distance from the tip rather than a high-intensity point, and scales more evenly with time.

Any field-shaping affects (such as with IFM) can be accounted for by adjusting the overall geometry of the O-field. Treatments in which a probe is intra-operatively displaced along its axis to produce a cylindrical lesion could also be captured by applying translation matrices to the O-field map at different time steps. These three (or possibly more) parameters may be combined to give each voxel a score at each time point. A simple formulation might be ε×O−I, although the actual algorithm will likely be more complex, and optimized by a validated math model. A lesion boundary may then be represented as an iso-surface where this value is equal to a given threshold.

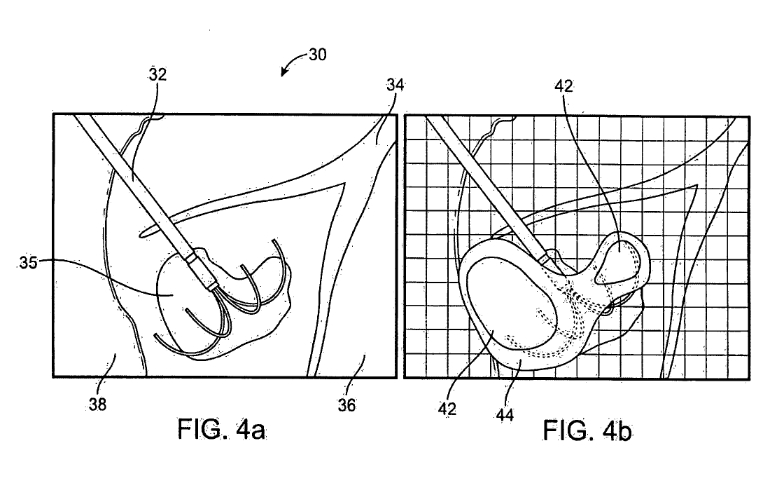

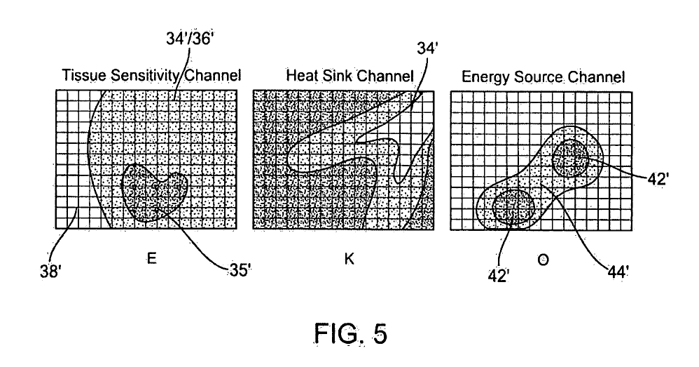

FIGS. 4A and 4B depict a rendering of a treatment site in the body during a simulated ablation procedure, and FIG. 5 shows a three-channel display communicating the relative magnitudes of each voxel <ε, K, O> at the site depicted in FIGS. 4A-4B. These five images may be simultaneously displayed on a computer display of the planning station. This information may communicate to a health professional the state of cell necrosis (as indicated by isosurfaces 42, 44) for the tumor 35 and the effects on neighboring tissue and/or anatomical bodies due to the presence of therapeutic energy source 32, which in this case is a multi-pronged RFA device. Referring first to FIG. 4A, which shows a rendered view of the site 30 and ablation tool 32 location, the site includes a first tissue type 36, the tumor 35, second tissue type 38, and vascular body 34. FIG. 4B illustrates the discretization of the site model and the state of ablation 42, 44. For example, isosurfaces 42, 44 may represent isotherms.

The isosurfaces depicted by 42, 44 are computed from the <ε, K, O> vector which may factor in safety margins (to account for such things as image quality relied on to perform an anatomic atlas mapping, discussed below, variations of tissue type, the presence of nearby vascular bodies, microcirculation, etc.) in order to protect adjacent tissue. FIG. 5 depicts the relative magnitudes of each voxel for the site depicted in FIG. 4A. Thus, the E-channel depicts the relative tissue sensitivity of the bodies and tissue to the heat source, the K-channel shows the heat sink qualities of the bodies and the o-channel the energy sources (element numbering 34/36′, 34′, 42′, 44′, 38′ and 35′ is intended to indicate the relationship between the structure in the rendering 30 and the isosurfaces). One or more of the channels of FIG. 5 may be superimposed over the renderings in FIG. 4A. The channels may be combined by an algorithm optimized by the simulation.

Error margins may be adjusted by adjusting a threshold. Qualitatively, the extent of the lesion could be increased or decreased based on the tissue sensitivity, and decreased locally based on the heat-sink characteristics of nearby blood vessels. Parameters of the simulation tool are adjusted such that the predictions of the algorithm will match the validated math model as closely as possible. Thus, the validated math model described earlier may form the basis of a reduced-parameter solution space, e.g., <ε, K, O> vector space approach, for rapid computation at the point of care. These parameters may be continuously updated and validated by the math model, experimental data, and/or comparison with pre- and post-operative anonymous image sets.

The accuracy of this type of algorithm will depend on the effects of different features in the treatment zone combining with linear or near-linear superposition. For example, a possible non-linear heat sink affect of multiple vessels near one another would need to be considered, e.g., a bifurcation. The goal is to be able to simulate field heating from microwave probes. In other embodiments, an EM absorption characteristic may be added to the <ε, K, O> vector space to account for the electrical properties of tissues.

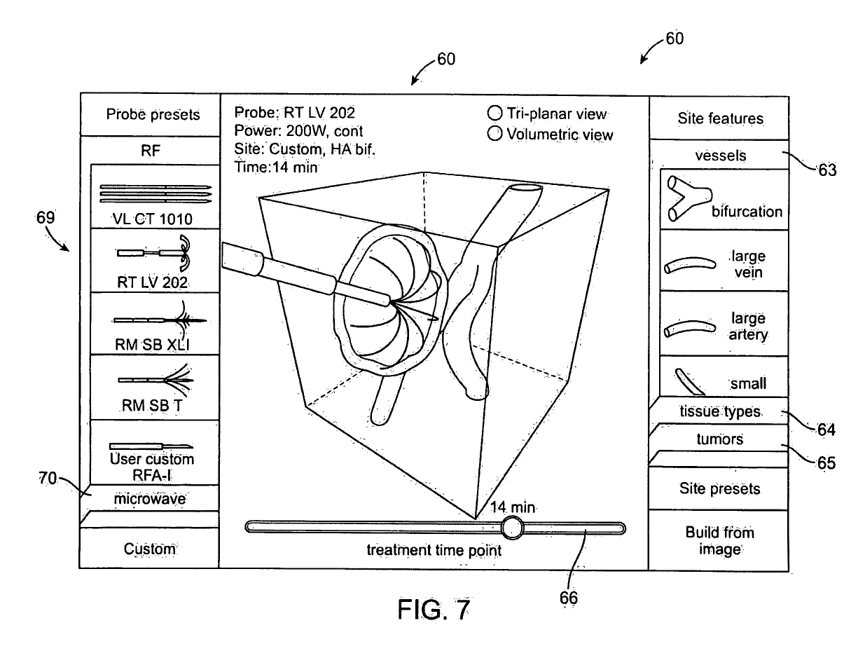

The predictive algorithm described above may be provided as a software package for interactive simulation of the effects of a given ablation procedure for the anatomical landscape of the patient. The package may include preset libraries of probes and pathology site locations, as well as a mechanism for allowing the user to create their own (as discussed in greater detail, below). The treatment site may be built from combining pre-packaged anatomical features such as vessels and tumors, or from a module which semi-automatically segments and processes patient image sets. The user may spatially manipulate the probe within the treatment space and receive real-time feedback of the thermal lesion extent at any given time point. An example of a GUI for one embodiment of this software product is depicted in FIG. 7.

The attributes of a software package developed in accordance with the above algorithms and model development described earlier provide accurate predictions based on validated physics-based models of thermal ablation in near real-time with respect to user manipulation of a virtual probe. Further, it will be easy to develop an intuitive graphics-based environment to construct a treatment site from pre-packaged anatomical features or a segmented CT, MR or US image. The outputs will also be easy to view, interpret and manipulate. The software routines can find solutions quickly, compute accurate ablation predictions, and may also include features such as treatment optimization (searching for best probe orientation or least number of ablation applications to treat a particular tumor) or probe path planning.

### Thermal Ablation Planning Station Examples

According to a third aspect of the disclosure, a software package forms part of a system for bedside care with a real-time pre and intra operative imaging suite that can dynamically update and optimize treatment.

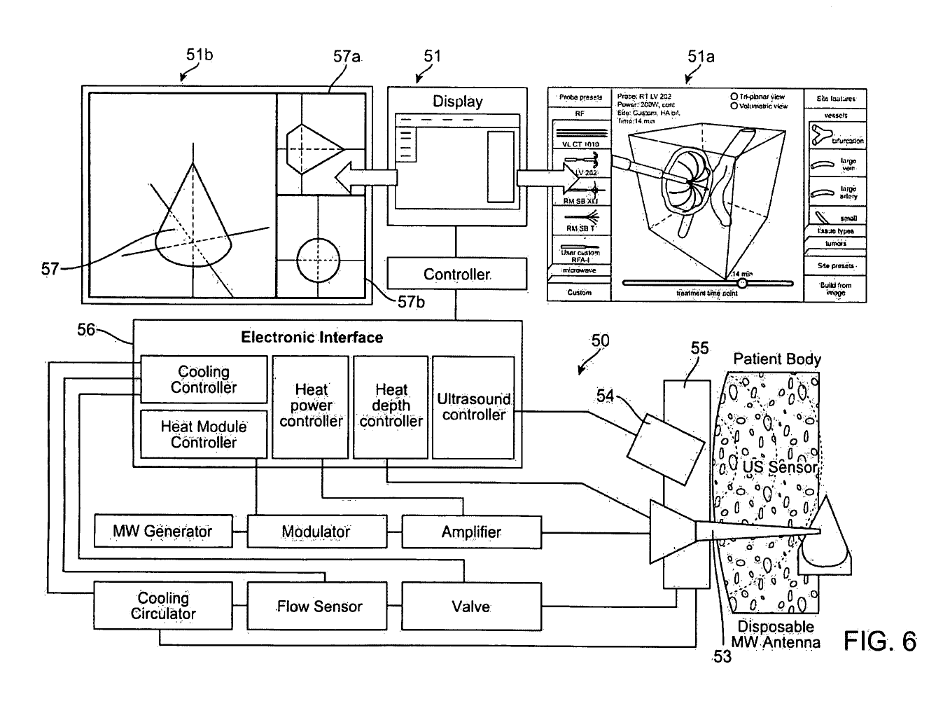

An example of such an ablation system is depicted in FIG. 6. The system includes a display 51 that shows images of the patient's interior near a tumor or other targeted tissue (51b) as generated from a three-dimensional scanning ultrasound sensor 54. This three-dimensional scanned image 51b provides volumetric and spatial information for bodies, such as a tumor 57. Through this image, the volume and shape of the tumor can be determined, as indicated by the screen shots 57a and 57b. The dimensional and spatial features may be identified by a point and select graphical tool as known in the art. This information may be used to construct a patient-specific simulation tool using a graphical model build environment 51a, as discussed in greater detail, below. The control of the probe 50, including its instrumentation parameters may be programmed through a planning tool GUI.

The system includes the MW antenna 53, US sensor 54, temperature sensor 55 and electronic interface 56 for this imaging and ablation device 50. The US sensor 54 is capable of generating timely and accurate 3D geometrical descriptions of a tumor and surrounding tissue as well. It will be understood that the foregoing is not limited to an IFM probe and US sensor for image generation. The planning station and probe features according to the IFM probe example may be readily applied to other thermal ablation technologies, such as RF, US and far-field MW. Further, imaging provided as part of the system in FIG. 6 may instead use a commercial 3D imaging system integrated with a probe planning station.

In one aspect of the disclosure, there is a need for an accurate assessment of a lesion's volume, which in connection with the example of FIG. 6 is dependent on, a) the calibration of US probe 54 to correct any spatial distortions inherit with US images, b) the correction methods for ultrasonic tissue propagation error, and c) intra-operative imaging protocols that would compensate for tissue deformations and breathing motions. An accurate assessment of the volumetric target (as an input to the system prototype) provides precise volumetric target accuracy during the treatment phase.

Based on the validated simulation models, a set of antenna and frequency configurations may be found that create predefined ablation volume elements. This information can be stored in a library and then recalled for the system in FIG. 6. Shape modeling algorithms may be used to process volumetric information obtained from an image-guidance part of the planning tool, e.g., as discussed earlier in connection with images 51b, which can proceed with breaking a pathology into multiple sub-segments of known geometries that would match a library of preset antenna signal configurations designed to encompass each sub-segment.

### Pre-Operative Planning and Inter-Operative Monitoring

The development of validated predictive physiological models of heat propagation for several common tissue formations and lesion characteristics was discussed earlier. The creation of such 4D predictive models lays the foundation for a preoperative thermal-ablation planning station, and an interoperative patient monitoring station. In accordance with a fourth aspect of the disclosure, a planning station provides a health care professional with a tool for creating patient-specific anatomical landscapes (“patient-specific model”), planning tools that operate over this model, and an ability to track patient progress using this patient-specific model.

Referring to FIG. 7, a GUI 60 for a planning tool includes a three-dimensional rendering and/or tri-planar rendering 67 of the patient-specific site 60. This rendering depicts the elements of the thermal simulation or EM and thermal simulation for RF probe types 69 or microwave probe types 70 over time 66. This patent-specific model may be constructed according to the following process. First, images of the site within the body are collected in order to obtain information about volumetric and spatial properties among anatomical bodies proximal a tumor. For instance, images 51b obtained using the three-dimensional US sensor 54 may be used to determine volumetric information 57a, 57b by a graphical drawing tool.

Using the 3D images, or MRI images, anatomical bodies are identified with bodies found in a library of anatomic structures, which relate to the validated math models discussed earlier. For instance, a menu system presents to the user a selection of different characteristic types of vessels 63, tissue types 64, and tumor structures 65. Each of these selections may correspond to particular model, or the aggregate of the shapes selected may be associated with a model containing the selected collection of sub-anatomical bodies. The orientation and relationship among the bodies may be performed graphically, e.g., by overlaying them on the image generated from US sensor. After this process is complete, the spatial relationships and volume information measured from the images is used with this model to form a patient-specific model by essentially morphing the constructed model onto the image of the patient site. This morphing may be accomplished by an anatomic atlas mapping of the bodies identified in the patient image with the models from the library. Such a technique has been used previously in connection with 3D models of anatomical structure. Once this mapping is complete, the constructed model may be used to perform patient-specific pre-operative planning, e.g., probe placement, depth, power settings, as well as intra-operative patient monitoring. This patient-specific model may be used in place of, or along with the rapid-simulation algorithm described earlier.

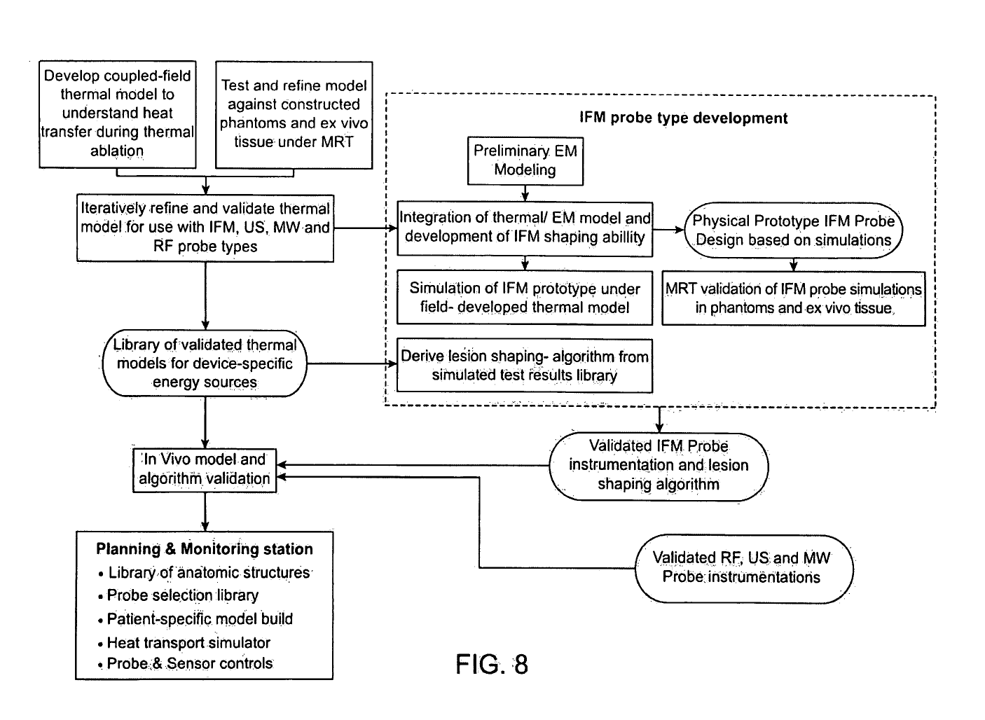

FIG. 8 depicts a flow diagram for a design of an IFM probe, a process for thermal model validation, and creation of a planning and monitoring station. Examples of each of the steps in the flow diagram are provided above.

The first step is the construction and validation of thermal models. This is done by proceeding with increasing complexity of the models as they are validated against phantoms, as depicted in FIG. 2. These models may then be used to validate prototype probes, and/or to simulate heat transport phenomena for existing probe types. For an IFM probe, the models are integrated with EM models and validated against phantoms. Once validated, the thermal/EM models form the basis for a prototype IFM probe design. The heat deposition patterns produced by an IFM prototype are used to validate the EM/thermal model. Again this is accomplished by the process of progressively increasing in complexity as discussed earlier. After this has been done, the models may be used to construct shaping algorithms so that probe characteristics (phase, position, frequency) can be manipulated to created shaped hot spots for treating tumors. A similar process may be followed for other probe types. The library of correlated math models, including reduced models with correlated vector quantities for fast simulations (e.g., <ε, K, O> voxels), the validated IFM probe information, and algorithms for optimizing the probe against particular types of anatomy, are combined to form a planning and monitoring system, or software package.

### EXAMPLE

Liposuction, a technique for removing blocks of fatty tissue, is considered by many to be cosmetic surgery. However, in the last few years, the liposuction technique has been incorporated into many facets of reconstructive surgery. Several noncosmetic uses for liposuction include: 1) undermining large flaps for reconstruction while preserving neurovascular attachments; 2) removal of lipomas; 3) treatment of gynecomastia; 4) contouring tissues after breast reconstruction; and 5) liposuction for improvement of axillary hyperhidrosis. Suction lipectomy, or liposuction, is the term used by plastic surgeons to describe the surgical disruption and removal of subcutaneous adipose tissue by means of a vacuum assisted cannula. A cannula is a narrow cylindrical tube with a blunted end and a suction port that is attached to a vacuum device. It is selected by its maximum diameter, ability to dissect through tissue, and the ease of fat removal, thus avoiding large surgical incisions. In addition to the negative pressure suction, the movement of the cannula facilitates fat removal by curettage of the fat aided by the manual squeezing of the fat. The movement of the cannula removes fat through the opening and can potentially cause trauma and hemorrhage to surrounding tissues. Several adaptations to the basic concept of liposuction have been advocated to improve results, minimize complications, and enhance the removal of adipose tissue. These adaptations include injections of low-dose epinephrine and local anesthetic to minimize bleeding and discomfort. Additionally, more efficient and less traumatic suction cannulas were developed. The “tumescent technique” of liposuction was introduced in 1986. The use of a multihole infiltration needle has allowed the anesthetic solution to be rapidly injected through the same incision used for liposuction. This has permitted the surgeon to efficiently anesthetize large subcutaneous areas while diminishing the need and risks of general anesthesia. Injection of a large volume of dilute lidocaine produces a swelling and firmness to the site that greatly facilitates fat removal. Over time, much larger volumes of lidocaine were administered, resulting in the capability of aspirating significantly greater volumes of tissue. All this was achieved with serum lidocaine levels that tests revealed were well below the toxicity range.

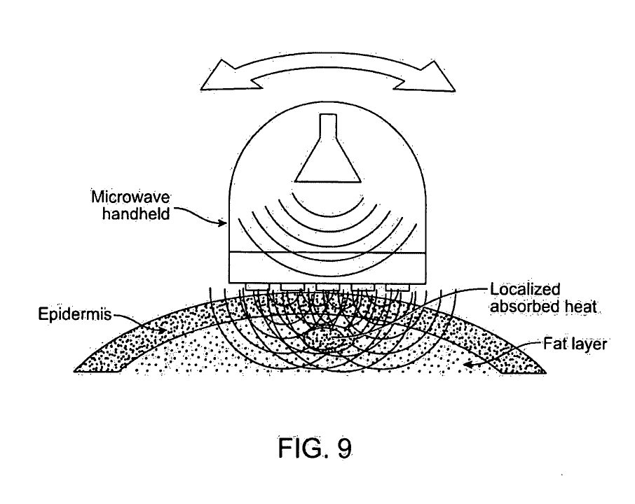

In this example microwave electromagnetic radiation is used to aid in adipose tissue reduction. The medical tool is designed to be manipulated by hand or an articulated arm over the patient's skin. This handheld device includes a monochromatic microwave radiating element (multiple antennas), a microwave beam shaper and a fat-layer sensor. The electromagnetic field delivered by the antennas is transmitted into interferential radiating waves. These interferential waves propagate through and under the patient skin. By constructive and destructive interference, electric field patterns are generated. Since the electrical field is heating tissue, a temperature gradient is generated around the pattern. One or multi-remote heat sources can be created in the depth of the patient body. This technology offers the advantages of a good contrast ratio between the constructive and destructive interference and to generate a remote heat source. Applying this interferometry technology over the skin of a fatty body part, a physician can heat, soften and even “burn” adipose layer without touching any surrounding tissue like the skin, dermis or muscles. FIG. 9 illustrates a handheld microwave device treating a fat layer just below the epidermis.

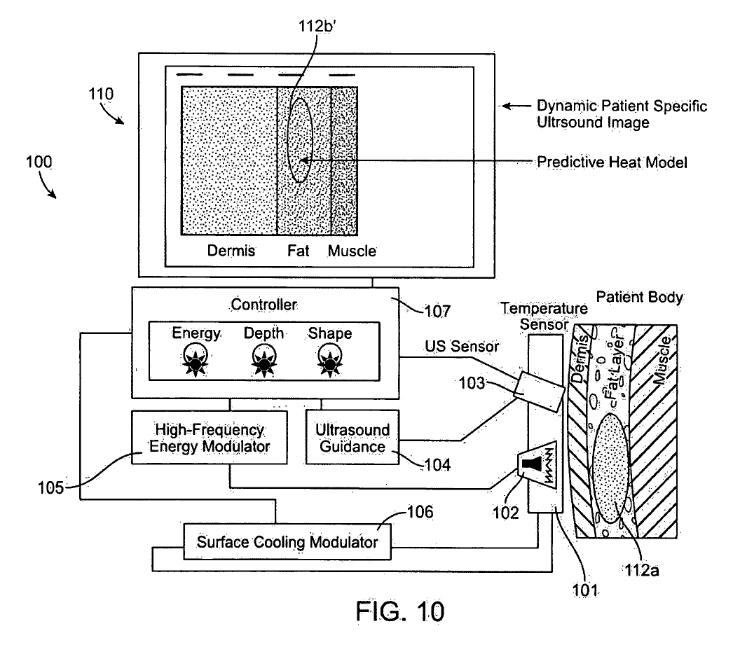

FIG. 10 is schematic illustration of the liposuction microwave probe system 100. The system includes a display portion 110, cooling element 101 and modulator 106, US imaging sensor 103 and associated guidance portion 104. A high-frequency modulator 105, operated by a controller 107, controls the spatial distribution of the microwave energy for generating the desired ablation 112b shape by a phase and frequency selection. The display 110 generates an image 112a of the shape based on the chosen antenna characteristics through controller 107. The shape 112a is overlayed on a real-time US image from US sensor 103. The controller 107 is depicted schematically as having a knob to control the MW energy level, and depth and shape for the heat spot.

The handheld probe system described above is integrated within a computer control system or station. The station is mobile and can be moved from one patient room to another. The handheld device (e.g., as depicted in FIG. 9) for system 100 includes temperature sensors to monitor a patient's skin temperature. As noted, the US sensor 103 generates real-time images of the region beneath the surface of the skin. This sensor may be an ultrasound linear or b-mode sensor which measures the patient's tissue profile. This type of imagery, registered spatially with the interferential element, provides the scaling of the layers necessary to determine the depth of where the heat should be applied and the shape of the hot spot. The microwave antenna 102 and the interferential element are mounted on a mobile platform that can be adjusted to set the proper heat depth of focus in the fat layer. The antenna probe may be oriented so as to minimize second order diffraction and optimize heat contrast.

The handheld probe is equipped with a cooling mechanism 101 to reduce heat generated at the handheld interface. The cooling mechanism 101 is built with a cooling circulator, a flow sensor and a valve. All three devices are computer controlled to optimize the temperature profile of the patient's body tissue. The fluid flow control, derived by the cooling circulator, regulates the heat absorption at the surface of the patient's skin and can be optimized to improve the heat contrast within the tissue. One can imagine changing the fluid temperature as well to optimize the temperature profile under the patient's skin. The temperature sensors are used as feedback mechanisms to adapt the fluid flow or fluid temperature.

The microwave is generated by a 2.45 GHz magnetron source. Other frequencies could be utilized depending on the precise nature of the procedure. The generated signal is then modulated to optimize the tissue heating contrast. The pulsed high-frequency can also increase the heat depth in living tissue. The modulated signal is then amplified to several hundred Watts and is delivered to the antennas. Then the probe radiates the electromagnetic field to creates interferential wavelets which are constructively focusing the near electromagnetic field at a dedicated distance or depth. The current generated in the conductive tissue is then heated at a precise spot to form pattern 112b′.

The liposuction procedure is non-invasive. A regular procedure may take several sessions to reduce the fat to a desired result. The different sequences related to the procedure are described below:

A caregiver places the handheld over the patient's body at the region to be treated for fat reduction. The depth of different tissue layers thickness are then verified using the US images. The apparatus is scanned over the adipose area to verify that the tissue profile matches the apparatus settings. The handheld device may be adjusted to match a particular patient tissue profile (i.e., different interferential or harmonics element, adjust diffractive element to patient skin distance, etc.). A marker may be used to delineate the body region where the fat reduction procedure is needed. The device's electronic real time feedback is implemented (LEDs, tissue profile graphic, layer thickness display). The probe's microwave power is then energized and the probe scanned over the region to be treated. The scanning may be performed in a systematic fashion with a regular and specific scanning speed. An integral part of the system is the heat feed-back and monitoring mechanism. This mechanism allows the caregiver to adjust the shape, amount and depth of the energy being implemented. These parameters may be adjusted dynamically based on the displayed ultrasound guidance image and heat-monitoring feedback received during the procedure. A thin layer of fat (1×1×4 cm3) may be progressively heated. The procedure finishes when the fat layer has reached a predetermined and recommended thickness. The treated fat (˜500 cc/treatment) will be naturally eliminated, re-absorbed by the patient body after a predetermined period of several weeks. Several fat reduction procedures can be carried out depending on the fat layer thickness.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A method for providing a health professional with a patient-specific thermal ablation software and apparatus, comprising the steps of:
constructing mathematical site models simulating heat transport phenomena within the body; and
providing the health professional with a machine based routine for predicting the progress of a patient's thermal ablation session using a simulation based on one or more of the site models.

2. The method of claim 1, further including the step of replacing or updating the site models with one or more database models based on, or acquired from a validation of each constructed site model including validating the site models against constructed phantoms.

3. The method of claim 2, wherein the database includes models based on in vivo data, in vitro data, and/or data based on structure that mimics physical anatomy.

4. The method of claim 1, wherein the validating step is based on progressive degrees of model complexity, including the steps of validating a first version of a model against a corresponding first phantom, and then validating a second, more complex second model against a corresponding phantom using the results of the first step to construct the second model.

5. The method of claim 2, wherein the constructing and validating steps includes the steps of
applying energy to the site phantom while measuring the temperature of the site phantom at one or more locations, and
correlating the site model with the site phantom including at least evaluating the accuracy of the modeled energy absorption and dissipation characteristics based on the one or more measured temperatures.

6. The method of claim 1, wherein the simulation is over a simplified vector space comprising computing parameters reflecting qualitatively the state of ablation.

7. The method of claim 6, wherein the reduced vector space comprises a tissue sensitivity, heat source and heat sink voxel distribution over a Cartesian space.

8. The method of claim 1, wherein the site models include models of sub-anatomical structures within the body including organs, vascular bodies and tumors.

9. The method of claim 1, wherein the site models include material properties that are both time and EM frequency dependant.

10. The method of claim 1, wherein the site models include models of vascular bodies.

11. The method of claim 1, wherein the site models include models of blood perfusion proximal a tumor.

12. The method of claim 11, wherein the thermal properties of modeled tissue are both time and temperature dependant.

13. The method of claim 1, wherein the site models are integrated thermal and EM models such that a simulation is run over the model based on an input control parameter for a probe.

14. The method of claim 13, wherein the probe is a near field interferential microwave probe.

15. A thermal therapy tool provided on computer-readable media, comprising:
a library of generalized site models, wherein each site model includes a mathematical representation of energy absorption and dissipation characteristics of various inhomogeneous tissue and/or anatomic structure within the body;
a first routine constructing a patient-specific model from one or more library models based upon patient-specific input parameters, the patient-specific parameters reflecting the nature of, and relationship among anatomical structures proximal to a site targeted for thermal therapy;
a second routine for simulating a thermal response at the targeted site, the routine receiving as input one of a plurality of user-selectable energy sources and the patient-specific model; and
a third routine for generating a visual representation of the predicted thermal response within a patient for thermal ablation planning and/or monitoring.

16. The thermal therapy tool of claim 15, wherein the user-selectable energy sources include at least one of near-field phased microwave inferential energy source, an ultrasound device and a radio-frequency device.

17. The thermal therapy tool of claim 15, wherein the patient-specific model is correlated with an anatomic atlas model of a generalized model onto the patient site.

18. The thermal therapy tool of claim 17, wherein the patient-specific model is constructed using an anatomic atlas mapping routine which takes as input a generalized model and coordinates of the patient site.

19. The thermal therapy tool of claim 17, the first routine further including a routine for generating a mapping from a 3D ultrasonic image of a patient.

20. A method for patient thermal ablation planning for patient-specific anatomy, comprising the steps of:
providing validated mathematical models anatomic structure; and
constructing a patient-specific model based on imaged patient-specific anatomy using an anatomic atlas mapping of one or mathematical models onto the patient-specific anatomy.

21. A near-field interferential microwave ablation system, comprising
a probe comprising a plurality of antennas for generating an energy pattern based on near-field interferential microwaves;
a controller for modifying a phase and frequency of one or more of the antennas; and
a planning station for the probe configured to identify the phase and frequency of the one or more antennas necessary to create a desired thermal ablation shape, wherein the phase and frequency are identified from results of a simulation using predictive models.

22. The near-field interferential microwave ablation system of claim 21, wherein the algorithm is based on validated mathematical models comprising an integrated thermal and EM model.

23. The near-field interferential microwave ablation system of claim 21, wherein the algorithm computes a schedule of antenna phasing, frequency of EM waves, and placement of the probe to create a three-dimensional ablation shape.

24. The near-field interferential microwave ablation system of claim 23, wherein the shape is selected based on characteristic tumor shapes.

25. A method for monitoring the thermal therapy applied to a diseased site within a patient's body, comprising the steps of:
providing a patient-specific heat model for predicting the energy absorption and dissipation properties of inhomogeneous tissue characteristic of the diseased site;
providing as input to the patient-specific heat model the treatment parameters including the type of device being used to supply energy to the diseased site; and
contemporaneously simulating the thermal response at the diseased using the heat model.

26. A method for monitoring a thermal ablation procedure, comprising the steps of
defining a set of parameters reflecting at least a thermal sensitivity, heat sink and heat source property of anatomical structure;
computing from the parameters a scoring for assessing the progress of the thermal ablation procedure; and
displaying a real-time depiction of the degree of tissue necrosis state relative to a device-specific probe.
